(19) United States
(12) Patent Application Publication
(10) Pub. No.: US 2026/0290524 A1
(43) Pub. Date: Sep. 24, 2026

(54) REMOTE MEDICAL MONITORING

(71) Applicant: Maquet Critical Care AB, Solna (SE)

(72) Inventor: Mari Andersson, Visby (SE)

(21) Appl. No.: 19/476,132

(22) PCT Filed: Apr. 16, 2024
(86) PCT No.: PCT/SE2024/050363

§ 371 Date: Oct. 16, 2025

(30) Foreign Application Priority Data
Apr. 17, 2023 (SE) 2350458-2

(51) Int. Cl. G16H 10/60; G16H 40/67

(52) U.S. Cl. CPC G16H 10/60; G16H 40/67

(57) ABSTRACT

The disclosure relates to a system for remote monitoring of a patient located at a patient site of a healthcare facility. The system communicates patient data obtained at the patient site to a remote display device, and displays the patient data as real-time patient data on a display of the remote display device as long as the patient data can be communicated to the remote display device in near real time. When a real-time data disruption during which the patient data cannot be communicated to the remote display device in near real-time occurs, the system is configured to display pre-disruption patient data corresponding to patient data obtained prior to the real-time data disruption on the display of the remote display device, as substitute for real-time patient data, and to visually indicate on the display of the remote display device that the pre-disruption patient data is not real-time patient data.

### TECHNICAL FIELD

The present disclosure pertains to the field of real-time remote patient monitoring. In particular, the present disclosure relates to a system, method and computer program for remote monitoring of a patient in a healthcare facility, as defined in the introductory parts of the appended independent claims.

### BACKGROUND

Remote patient monitoring (RPM) involves collection of patient data by means of an electronic medical device, and transmission of the patient data from the medical device to a remote healthcare provider who can then analyse the data and track the health of the patient remotely. Real-time RPM is a subset of RPM where the patient data is transmitted to the healthcare provider in real-time, so that the healthcare provider can monitor the patient's current health status and intervene if necessary.

Medical devices for collecting the patient data in a real-time RPM system may, for example, include wearables, sensors, medical treatment devices, local patient monitoring systems, and other monitoring tools that can track vital signs such as heart rate, blood pressure, oxygen levels, and respiratory rates.

Examples of medical treatment devices that may offer remote access to patient data obtained during ongoing treatment include mechanical ventilators. Today, many leading ventilator manufactures offer products that can be used to give a remote clinician access to patient data obtained by the ventilator during ongoing mechanical ventilation via a remote display or monitor. Such patient data may relate both to the patient and to the operation of the ventilator. A few solutions even enables remote control of the ventilator via the remote display or monitor.

Solutions for remote patient monitoring often require data communication over wireless networks and are thus prone to downtime due to disruption or loss of wireless network connectivity. Most remote monitoring solutions are incapable of providing access to patient data during such a disruption in network connectivity.

Consequently, disruptions in network connectivity may prevent display of patient data to the remote clinician or device operator. It may also cause outdated patient data to be displayed to the remote clinician or medical device operator which, in turn, may cause the clinician or device operator to take patient-critical decisions based on outdated and sometimes misleading patient data. This is a concern for patient safety, in particular during real-time RPM of critically ill patients.

U.S. Pat. No. 1,121,116 and U.S. Pat. No. 9,041,532 disclose examples of systems that, at least to some extent, relate to remote medical monitoring and are concerned with the problem of disruption or loss of network connectivity.

Still, in view of state-of-the-art systems for remote medical monitoring, there is a need to overcome or at least mitigate some of the problems caused by a disruption in network connectivity or any other phenomenon preventing patient data to be communicated from the medical device at the patient site to the remote display or monitor.

### SUMMARY

It is an object of the present disclosure to mitigate, alleviate or eliminate one or more of the above-identified deficiencies and disadvantages in the prior art.

In particular, it is an object of the present disclosure to provide means for facilitating decision making for a remote clinician remotely monitoring a patient, and especially for preventing that patient-critical decisions are taken based on outdated and potentially misleading patient data. Ultimately, it is an object of the present disclosure to improve patient safety during such remote monitoring.

According to a first aspect of the present disclosure there is provided a system for remote monitoring of a patient located at a patient site of a healthcare facility, such as a hospital or a medical clinic. The system is configured to communicate patient data obtained by a medical device at the patient site to a remote display device, and to display the patient data as real-time patient data on a display of the remote display device. The system is configured such that, when the patient data can be communicated to the remote display device in near real time, the patient data is displayed as real-time patient data on the display of the remote display device. On the other hand, when a real-time data disruption during which the patient data cannot be communicated to the remote display device in near real-time occurs, the system is configured to display pre-disruption patient data corresponding to patient data obtained prior to the real-time data disruption on the display of the remote display device, as substitute for real-time patient data. The system is further configured to visually indicate on the display of the remote display device that the pre-disruption patient data is not real-time data.

Patient data is often relevant for clinical decision making also for some time after it has been obtained. Some types of patient data change rarely or slowly and may stay relevant for a long time, whereas other types of patient data change often or quickly and therefore become outdated as a basis for clinical decision making very soon after it has been obtained. By displaying pre-disruption patient data corresponding to patient data obtained before the real-time data disruption and visually indicating that the pre-disruption patient data is not real-time data, the remote clinician is provided with potentially relevant patient data even during real-time data disruptions while at the same time being notified that the displayed patient data is old and therefore potentially unsuitable as a basis for clinical decision making.

That the pre-disruption patient data is displayed as substitute for real-time patient data means that the pre-disruption patient data is displayed in a manner similar to the real-time patient data on the remote display device. In some embodiments, it means that the pre-disruption patient data, upon a real-time data disruption, takes the place of the real-time patient data in a current view of a graphical user interface (GUI) of the remote display device.

The pre-disruption data that is displayed as substitute for the real-time data during the real-time data disruption is preferably the last obtained patient data that is communicable to the remote display device.

According to some embodiments, the system is configured to display, together with the pre-disruption patient data, information indicating when the pre-disruption patient data was obtained at the patient site on the display of the remote display device.

This way, the clinician remotely monitoring the patient is further assisted in clinical decision making based on the displayed pre-disruption patient data.

According to some embodiments, the system is configured to display the pre-disruption patient data on the display of the remote display device only during a predetermined time window.

This way, the risk that the remote clinician takes an erroneous clinical decision based on outdated patient data that may be irrelevant or even misleading is further reduced. Also, the GUI of the remote display device becomes more user friendly since other data and information displayed on the GUI is not obscured by outdated patient data. The duration of the time window may be set to correspond to a time during which the pre-disruption patient data can be assumed to substantially correspond to current patient data. This time may vary based on the type of patient data that is being displayed and may, for example, be determined based on the tendency of the patient data to change over time, a criticality of correctness of the patient data in clinical decision making, etc.

According to some embodiments, the duration of the time window is different for different types of patient data. This way, the time during which pre-disruption patient data is displayed may be individually adjusted for different types of patient data. For example, time windows for patient data types that stay relevant for a long time may have longer durations than time windows for patient data types that quickly becomes irrelevant or misleading.

According to some embodiments, the system is configured to display the real-time patient data and the pre-disruption patient data in a real-time view of a graphical user interface, GUI, of the remote display device, and, when the predetermined time window has elapsed, prevent further display of pre-disruption patient data as substitute for real-time patient data in the real-time view. The system may, however, be configured to still allow the pre-disruption patient data to be displayed as historical patient data in a non real-time view of the GUI.

This is advantageous in that the pre-disruption patient data becomes available to the remote clinician also after the time window has elapsed while minimizing the risk that the clinician mistakes the pre-disruption data for real-time data. The historical patient data may, e.g., comprise trends or trend curves for the pre-disruption patient data, which is often more relevant in clinical decision making than one or more distinct patient data points obtained some time ago.

The medical device obtaining the patient data at the patient site may be any type of electronic medical device capable of obtaining patient data relating to a patient and/or an ongoing medical treatment of the patient and to communicate the patient data to the remote display device either directly or via other devices or network nodes. Non-limiting examples of such medical devices include wearable medical devices, sensors, medical treatment devices, local (on patient site) patient monitoring systems.

In cases where the medical device is associated with a graphical user interface (GUI), the remote display device may advantageously be provided with a GUI that is a replica of the GUI associated with the medical device.

Designing the GUI of the remote display device as a replica of the GUI associated with the medical device makes the remote clinician, who is typically an experienced user of the medical device and therefore familiar with the GUI associated with the medical device, capable of navigating the GUI of the remote display device without further training. Also, and more importantly, it makes it easier for the clinician to perceive the visual indication indicating that the pre-disruption patient data is not real-time data. This is because a clinician who is used to handle and operate the medical device is used to the GUI of the medical device and hence to the design of any GUI view in which real-time patient data is presented. A visual indication indicating that the presented patient data is not real-time data, which indication is not normally presented in this GUI view, will therefore catch the attention of the clinician more easily than if the clinician would not have been used to the GUI.

According to some embodiments, the system comprises a server configured to receive the patient data obtained at the patient site and communicate the patient data to the remote display device for display as real-time patient data.

In some embodiments, the obtained patient data may be transmitted directly to the remote display device by the medical device or any other device at the patient site to which the medical device is communicatively connectable. However, using an intermediate server that receives the obtained patient data and communicates the patient data to the remote display device brings about several advantages in terms of system architecture and functionality. For example, it allows patient data to be buffered and/or saved centrally in the server, and facilitates access to patient data from different remote display devices.

According to some embodiments, the server is configured to provide the functionality described herein via a web application that is stored on the server and accessible by the remote display device via a web browser of the remote display device. This allows any web browser-equipped display device to serve as a remote display device for remote monitoring of patients, in accordance with the teachings of the present disclosure.

According to some embodiments, the system is configured to save the received patient data in the server and, upon a real-time data disruption preventing the server from receiving patient data from the patient site but allowing the server to communicate with the remote display device, to communicate the saved patient data to the remote display device for display as historical patient data in the non real-time view of the GUI. This allows pre-disruption patient data to be displayed as historical patient data by the remote display device without having to store patient data in the remote display device itself.

According to some embodiments, the server is configured to buffer, for a predetermined buffer time, the near real-time patient data in the server, and to transmit the buffered near real-time patient data to the remote display device as near real-time patient data to be displayed on the display of the remote display device. This way, differences in the rate at which patient data is transmitted from the patient site and the rate at which the patient data can be received in the remote display device can be compensated for.

According to some embodiments, the system is configured to receive, in the server, patient data obtained at a plurality of patient sites, each hosting a respective patient, and simultaneously display patient data obtained at the plurality of patient sites as real-time patient data on a display of a single remote display device communicatively connected to the server. Such a system for remote and simultaneous monitoring of multiple patients is advantageous in that patient data from multiple patient sites can be easily compared by a remote clinician. Such a comparison facilitates detection of deviations in patient data, which deviations may otherwise be hard to detect. In some embodiments, this remote multi-monitoring system may be configured to display a dashboard view comprising replicas of the GUIs of the medical devices of the plurality of patient sites, presented side by side on the display of the remote display device. This allows the remote clinician to compare patient data presented in the same or a similar manner, which further facilitates detection of patient data deviations. The dashboard view of the remote display device may be controlled by the remote clinician to present real-time views or non real-time views of the GUI replicas, in order for the remote clinician to compare any of real-time patient data or historical patient data, e.g. presented in the form of trends or trend curves, between the different patient sites.

As mentioned above, the medical device obtaining the patient data at the patient site may be any type of electronic medical device. In some embodiments, the medical device is a medical device providing critical care treatment to the patient at the patient site, such as a mechanical ventilator providing respiratory treatment to the patient, or an extracorporeal membrane oxygenation (ECMO) device providing ECMO treatment to the patient. In other embodiments, the medical device may be a bedside patient monitor for bedside monitoring of a physiological status of the patient, or a wearable medical device configured to be worn on the body of the patient.

According to a second aspect of the present disclosure there is provided a method in a system for remote monitoring of a patient located at a patient site of a healthcare facility, wherein the system is configured to communicate patient data obtained by a medical device collocated with the patient at the patient site to a remote display device, and to display the patient data as real-time patient data on a display of the remote display device. The method comprises the steps of, when the patient data can be communicated to the remote display device in near real time, displaying the patient data as real-time patient data on the display of the remote display device, and, when a real-time data disruption during which the patient data cannot be communicated to the remote display device in near real-time occurs, displaying, on the display of the remote display device, pre-disruption patient data corresponding to patient data obtained prior to the real-time data disruption, as substitute for real-time patient data, and visually indicating on the display of the remote display device that the pre-disruption patient data is not real-time data.

According to some embodiments, the method comprises the step of displaying on the display of the remote display device, together with the pre-disruption patient data, information indicating when the pre-disruption patient data was obtained at the patient site.

According to some embodiments, the pre-disruption patient data is displayed on the display of the remote display device only during a predetermined time window.

According to some embodiments, the real-time patient data and the pre-disruption patient data is displayed in a real-time view of a GUI of the remote display device, wherein the method comprises the steps of, when the predetermined time window has elapsed, preventing further display of pre-disruption patient data as substitute for real-time patient data in the real-time view, optionally while allowing pre-disruption patient data to be displayed as historical patient data in a non real-time view of the GUI.

According to some embodiments in which the medical device is associated with a GUI, the GUI of the remote display device is a replica of the GUI associated with the medical device.

According to some embodiments, the method comprises the steps of receiving, in a server, the patient data obtained at the patient site, and communicating the patient data to the remote display device for display as real-time patient data.

According to some embodiments, the method comprises the steps of saving the received patient data in the server and, upon a real-time data disruption preventing the server from receiving patient data from the patient site but allowing the server to communicate with the remote display device, communicating the saved patient data to the remote display device for display as historical patient data in the non real-time view of the GUI.

According to some embodiments, the method comprises the steps of receiving, in the server, patient data obtained at a plurality of patient sites, each hosting a respective patient connected to a medical device for obtaining patient data from the patient, and simultaneously displaying patient data obtained at the plurality of patient sites as real-time patient data on a display of a single remote display device communicatively connected to the server.

The method is a computer-implemented method that is performed by the system upon execution of a computer program by one or more processors of the system.

Thus, according to a third aspect of the present disclosure, there is provided a computer program for a system for remote monitoring of a patient located at a patient site of a healthcare facility, wherein the system is configured to communicate patient data obtained by the medical device at the patient site to a remote display device, and to display the patient data as real-time patient data on a display of the remote display device. The computer program comprises computer-readable instructions which, when executed by at least one processor of the system, causes the system to perform the above-described method.

According to some embodiments, the computer program is a web application residing in the server, accessible by the remote display device via a web browser of the remote display device.

In other embodiments, the principles of the present disclosure may be implemented by means of a distributed computer program, such as a client-server application. In this scenario, the method may be performed upon execution of a client application residing in the remote display device, which client application is configured to communicate with a server application residing in the server.

In yet other embodiments, the method may be performed upon execution of first computer program component residing in the remote display device, which first computer program communicates with a second computer program component residing in the medical device, whereby the patient data obtained at the patient site may be transmitted directly to the remote display device, without any intermediate server.

According to a fourth aspect of the present disclosure there is provided at computer program product comprising a data storage, such as a non-transitory memory hardware device, storing the above-mentioned computer program.

Effects and features of the second, third and fourth aspects are to a large extent analogous to those described above in connection with the first aspect.

Other effects and advantages of the system, method and computer program of the present disclosure will become apparent from the detailed description following hereinafter. The detailed description and its specific examples disclose preferred embodiments of the disclosure by way of illustration only. Those skilled in the art understand from guidance in the detailed description that changes and modifications may be made within the scope of the disclosure.

Hence, it is to be understood that the disclosure is not limited to the particular component parts of the devices described or steps of the methods described since such devices and method steps may vary. It is also to be understood that the terminology used herein is for purpose of describing particular embodiments only, and is not intended to be limiting. It should be noted that, as used in the specification and the appended claim, the articles “a”, “an”, “the”, and “said” are intended to mean that there are one or more of the elements unless the context explicitly dictates otherwise. Thus, for example, reference to “a unit” or “the unit” may include several devices, and the like. Furthermore, the words “comprising”, “including”, “containing” and similar wordings does not exclude other elements or steps.

### BRIEF DESCRIPTIONS OF THE DRAWINGS

The above objects, as well as additional objects, features and advantages of the present disclosure, will be more fully appreciated by reference to the following illustrative and non-limiting detailed description of example embodiments of the present disclosure, when taken in conjunction with the accompanying drawings.

FIG. 1 illustrates a system for remote monitoring of a patient located at a patient site, according to an exemplary embodiment of the present disclosure.

FIG. 2 illustrates an example of a real-time view of a graphical user interface (GUI) presenting real-time patient data from the patient site on a display of a remote display device.

FIG. 3 illustrates an example of the real-time view of the GUI in the event of a real-time data disruption during which pre-disruption patient data is substituted for real-time patient data in real-time patient data fields of the real-time view.

FIG. 4 illustrates an example of the real-time view of the GUI when the real-time data disruption has lasted for certain period of time exceeding a duration of a predetermined time window, whereafter display of pre-disruption patient as substitute for real-time patient data is prevented.

FIG. 5 illustrates an example of a non real-time view of the GUI, which view provides access to pre-disruption patient data presented as historical patient data also during long-term real-time data disruptions.

FIG. 6 illustrates a system for remote monitoring of multiple patients located at different patient sites, according to an exemplary embodiment of the present disclosure.

FIG. 7 is a flowchart illustrating a method performed in a system for remote monitoring of a patient located at a patient site, according to an exemplary embodiment of the present disclosure.

### DETAILED DESCRIPTION

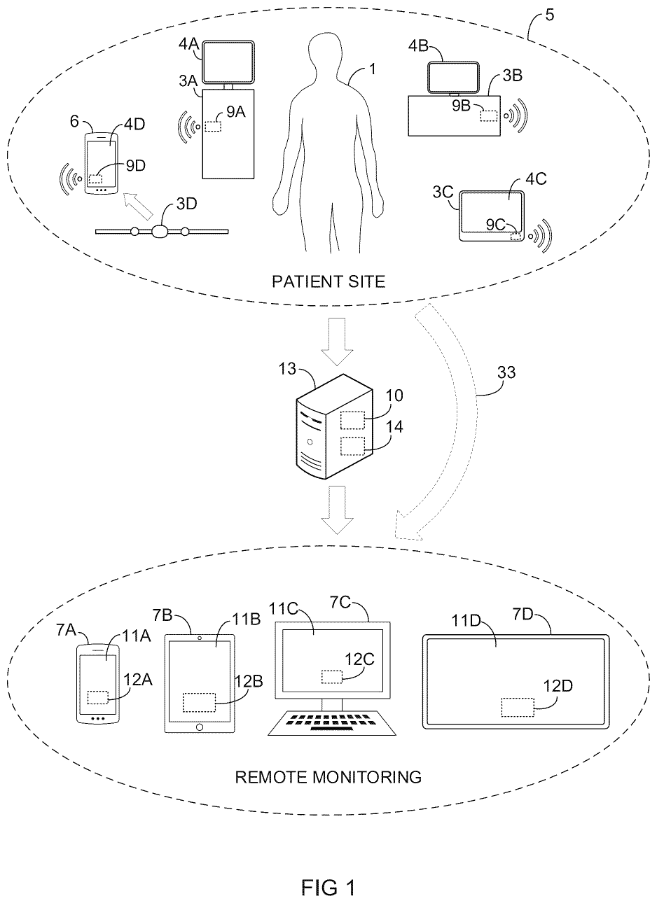

FIG. 1 illustrates an exemplary embodiment of a system for remote medical monitoring of a patient 1 located at a patient site 5 of a healthcare facility, such as a hospital or a medical clinic. The patient 1 is connected to a medical device 3A-D that is co-located with the patient 1 at the patient site 5 and configured to obtain patient data related to the patient 1 and/or an ongoing medical treatment of the patient. The system is a remote patient monitoring (RPM) system for near real-time monitoring of the patient 5 from a location that is remote from the patient site 5 and may hereinafter sometimes be referred to as a real-time RPM system.

The medical device 3A-D may be any type of electronic medical device configured to obtain patient data at the patient site and facilitate communication of the patient data from the patient site to a remote display device 7A-D. The medical device 3A-D may be a medical-grade device specifically adapted and certified for use in hospital environments.

In some embodiments, the medical device 3A-D is a critical care treatment device, i.e. a medical treatment device configured to provide critical care treatment to the patient 1. For example, the medical device may be a breathing apparatus for providing respiratory treatment to the patient 1, such as a mechanical ventilator 3A or an anaesthesia machine. According to another example, the medical device is a heart-lung machine for oxygenation of the blood of the patient 1, such as an extracorporeal membrane oxygenation (ECMO) device 3B for providing ECMO treatment to the patient.

In other embodiments, the medical device 3A-D may be a bedside patient monitoring system 3C that is co-located with the patient at the patient site 5. In yet other embodiments, the medical device 3A-D may be a wearable medical device 3D that is worn on the body of the patient 1. In the illustrated non-limiting example, the wearable medical device 3D is a wearable electrocardiogramonitor.

The medical device 3A-D is configured to obtain patient data relating to the patient 1 and/or the operation of the medical device, typically using one or more sensors for measuring patient parameters or internal states and/or settings of the medical device.

In embodiments where the medical device is a breathing apparatus, such as a mechanical ventilator 3A, non-limiting examples of patient data that may be obtained by the medical device include an airway pressure (Paw) of the patient, a positive end-expiratory pressure (PEEP) applied to the airways of the patient, a respiratory rate (RR) of the patient, a minute ventilation (MV) of the patient, a tidal volume (VT) of breathing gas inspired and/or expired by the patient, an inspiratory to expiratory ratio (I:E) of the patient, an oxygen concentration of the breathing gas delivered to the patient, a carbon dioxide concentration of expiration gas exhaled by the patient, a dynamic compliance (Cdyn) of the patient, a heart rate, a mean arterial blood pressure (MAP), a body temperature, a blood oxygen saturation level and a central venous pressure (CVP).

In embodiments where the medical device is a heart-lung machine, such as an ECMO device 3B, non-limiting examples of patient data that may be obtained by the medical device include a flow rate of sweep gas supplied to an oxygenator of the ECMO device, pre-and/or post oxygenator oxygen and/or carbon dioxide concentrations, a pressure drop over the oxygenator, a gas transfer resistance of the oxygenator, blood parameters determined using a blood gas analyser (BGA) of the ECMO device, a venous and/or arterial pressure of the patient, a heart rate, a body temperature, a blood oxygen saturation level and CVP.

In embodiments where the medical device is a bedside patient monitoring system 3C, non-limiting examples of patient data that may be obtained by the medical device include a heart rate, ECG signals, a respiratory rate, a blood pressure, a body temperature and a blood oxygen saturation level.

In embodiments where the medical device is a wearable medical device 3D, non-limiting examples of patient data that may be obtained by the medical device include a heart rate, ECG signals, a respiratory rate, a blood pressure, a body temperature, a blood oxygen saturation level and a blood glucose level.

The medical device 3A-D may comprise or be connectable to a monitor or display device for presenting the obtained patient data to the patient 1 and/or a clinician who is co-located with the patient 1 at the patient site 5. For example, some medical devices 3A-C may comprise an integrated monitor 4A-4C for presenting obtained patient data to a clinician or medical device operator at the bedside of the patient 1. In the illustrated example, this applies to the ventilator 3A, the ECMO device 3B, and the bedside patient monitoring system 3C. Other medical devices may be coupled to an external display device at the patient site 5 to facilitate presentation of patient data obtained by the medical device on a display of the display device. In the illustrated example, this applies to the wearable medical device 3D, which is coupled to an external display device 6 comprising a display 4D for presenting the patient data to the patient 1 and/or a clinician who is co-located with the patient 1 at the patient site 5. In this example, the external patient-site display device 6 is a smart phone to which the wearable medical device 3D transmits obtained patient data via e.g. a Bluetooth connection.

The patient data may be displayed on the monitor 4A-C or the patient-site display device 6 using a graphical user interface (GUI) associated with the medical device, which GUI may be navigated by the bedside clinician and/or the patient to change views, change medical device settings, set alarms, etc. This GUI is hence designed and intended for patient-site monitoring of the patient data and will, therefore, hereinafter be referred to as the patient-site GUI.

The system is further configured to communicate the patient data obtained by the medical device 3A-D to the remote display device 7A-D. In the illustrated embodiment, the patient data is transmitted from the patient site 5 by a transmission module 9A-D of the medical device 3A-D or its associated external display device 6. The transmission module 9A-D may be integrated in the medical device or the external display device or it may be an add-on transmission module that can be connected to a communication port of the medical device or the external display device to provide the medical device or the external display device with network connectivity capabilities. The transmission module 9A-D is configured to transmit the patient data to a system server 13. The server 13 is configured to receive and process the patient data and to communicate the patient data to the remote display device 7A-D. The server 13 is preferably located within the healthcare facility.

The transmission module 9A-D is typically but not necessarily configured to communicate the patient data to the server 13 wirelessly, e.g. via a wireless network. The network used for transmission of patient data from the transmission module 9A-D to the server 13 may be the Internet or a healthcare provider IT network, such as a local area network (LAN) of a health care facility or a private wide area network (WAN) of a health care organization. Preferably, the network is a healthcare provider IT network, whereby the network and the server 13 may be adapted to meet specific requirements and regulations governing networks and servers managing patient data in a hospital environment, in order to ensure the security, privacy and integrity of sensitive medical information. The transmission module 9A-D may be configured to use any available wireless communication technology, such as a Wi-Fi technology, a 4G or 5G mobile network technology, or the like. It should also be noted that the present disclosure is not limited to wireless communication and that the principles for remote near real-time monitoring of a patient described herein are applicable also when patient data is communicated from the patient site 5 to the remote display device 7A-D partly or completely via a wired connection.

The server 13 may be a web server connected to the Internet in order to allow remote monitoring of the patient 1 via any type of Internet-connected display device. The server 13 comprises at least one processor 10 and may be configured to provide the functionality described herein upon execution by the at least one processor of a computer program stored in a data storage 14, such as a non-transitory memory hardware device, of the server 13.

In the illustrated example, the computer program stored in the server 13 is a web application that can be accessed via a web browser stored in a data storage 12A-D, such as a non-transitory memory hardware device, of the remote display device 7A-D. The patient data is hence received from the transmission module 9A-D by the server 13, and communicated to the display device 7A-D via the web application stored in the server 13. Of course, in other embodiments, several network nodes may be involved in the communication of the patient data from the patient site 5 to the remote display device 7A-D. For example, the system may comprise a web server for receiving and/or storing the patient data from the patient site 5, and a web application server for hosting and running the web application that communicates the patient data to the remote display device 7A-D.

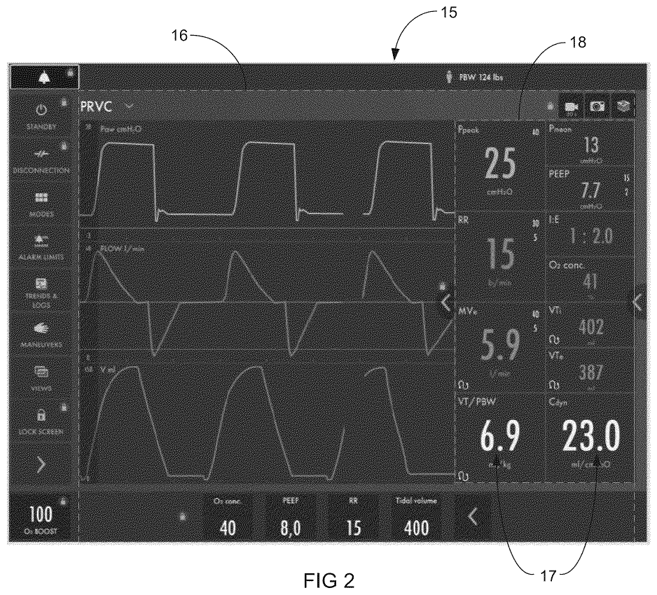

The system is configured to try to communicate the patient data obtained by the medical device 3A-D at the patient site 5 to the remote display device 7A-D in near real time, and, when the patient data can be communicated to the remote display device 7A-D in near real time, to display the patient data as real-time patient data on a display 11A-D of the remote display device 7A-D. When a real-time data disruption during which the patient data cannot be communicated to the remote display device 7A-D in near real-time occurs, the system is configured to display pre-disruption patient data corresponding to patient data obtained prior to the real-time data disruption on the display 11A-D of the remote display device 7A-D, as substitute for real-time patient data 17, and to visually indicate on the display 11A-D of the remote display device 7A-D that the pre-disruption patient data is not real-time data, as will be further described with reference to FIG. 2.

The term near real-time is normally defined as the timeliness of data or information which has been delayed by the time required for electronic communication and automatic data processing, which implies that there are no significant delays. This definition applies also to the present disclosure.

However, in some embodiments, the server 13 is configured to buffer the received patient data for a predetermined buffer time to compensate for differences in the rate at which patient data is transmitted from the transmission module 9A-D and the rate at which the patient data can be communicated to the remote display device 7A-D. The buffer time is the period of time during which patient data is allowed to build-up in the server 13 before being communicated to the remote display device 7A-D. Therefore, “near real-time” in the context of the present disclosure may be a buffer time for buffering patient data in the server 13, plus the time required for other data processing and communication steps in and between the medical device 3A-D, the server 13, and the remote display device 7A-D.

The buffer time may for example be 1-10 seconds, preferably 1-6 seconds, and most preferably 1-4 seconds. In a preferred embodiment, the buffer time is approximately 3 seconds. Since the buffer time typically constitutes the majority of the amount of time required to communicate the patient data to the remote display device 7A-D, this means that “near real-time” in the context of the present disclosure may correspond to a period of time of no more than 10 seconds, preferably no more than 6 seconds, and most preferably no more than 4 seconds. In the preferred embodiment, “near real time” means no more than 3 seconds. Furthermore, that the patient data is communicated to the remote display device 7A-D in near real-time means that the time from the obtaining of the patient data at the patient site 5 to the displaying of the patient data on the display 11A-D of the remote display device 7A-D does not exceed the above-mentioned time limits.

FIG. 2 illustrates a real-time view 16 of a GUI 15 of the remote display device 7A-D, which GUI is caused to be displayed on the display 11A-D of the remote display device 7A-D by the web application residing in the server 13, when run by the web browser of the remote display device. Strictly speaking, the GUI 15 is rather the GUI of the web application residing in the server 13 than a GUI of the actual remote display device 7A-D. However, throughout this disclosure, a GUI of the remote display device should be interpreted as any GUI that is presented on the display 11A-D of the remote display device 7A-D.

The real-time view 16 comprises at least one real-time data field 18 for displaying real-time patient data 17. The real-time patient data 17 is displayed in the at least one real-time data field 18 in a characteristic way, clearly identifying the displayed data as real-time data that has been obtained at the patient site 5 in near real time. In the illustrated example, real-time data is displayed either in colour or in white, against a black background.

The GUI 15 of the remote display device 7A-D is preferably a replica of the above-mentioned patient-site GUI associated with the medical device 3A-D. That the GUI 15 of the remote display device 7A-D is a replica of the patient-site GUI means that at least in the real-time view 16 in which real-time patient data is presented, the patient data is presented in the same or a similar way as in a corresponding real-time view of the patient-site GUI associated with the medical device 3A-D. Preferably, most views, menus, controls and other GUI objects of the patient-site GUI are replicated and presented in the same or similar way on the GUI 15 of the remote display device 7A-D as on the patient-site GUI. This effectively makes the remote display device 7A-D look and to most extent behave like the monitor 4A-C or the associated display device 6 of the medical device 3A-3D and the remote display device 7A-D may, in that sense, be turned into a “twin” of the medical device monitor 4A-C or associated patient-site display device 6 by running the web application provided by the system server 13. In the illustrated example, with simultaneous reference to FIG. 1, the GUI 15 is a replica of the GUI of the mechanical ventilator 3A, presented on the ventilator monitor 4A.

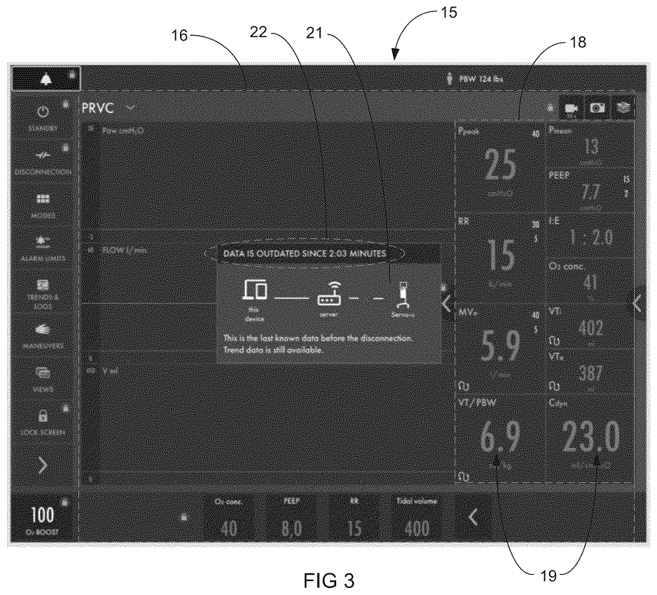

FIG. 3 illustrates the real-time view 16 of the GUI 15 during a first period of a real-time data disruption during which real-time patient data cannot be communicated to the remote display device 7A-D.

A real-time data disruption may be any event or phenomenon that prevents the patient data obtained by the medical device 3A-D at the patient site 5 to be communicated to the remote display device 7A-D in near real time. Typically, with the exemplary system architecture illustrated in FIG. 1, a real-time data disruption may be caused by wireless connection problems preventing the transmission module 9A-D from transmitting the patient data to the server 13.

During the first period of the real-time data disruption, which period typically starts as soon as the real-time data disruption has been detected by the system, the system is configured to display pre-disruption patient data 19 as substitute for real-time patient data 17 in the real-time view 16 of the GUI 15. More specifically, the system is configured to display the pre-disruption patient data 19 as substitute for the real-time patient data 17 by substituting the pre-disruption patient data 19 for the real-time patient data 17 in the at least one real-time data field 18 of the real-time view 16.

Furthermore, the system is configured to visually indicate in the real-time view 16 that the pre-disruption patient data 19 is not real-time patient data 17. This may be achieved by visually presenting the pre-disruption patient data 19 in a way that is different from the characteristic way in which real-time patient data 17 is presented in the real-time view 16. Instead or in addition to changing the visual appearance of the displayed patient data when switching from real-time patient data 17 to pre-disruption patient data 19, the visual indication indicating that the pre-disruption patient data 19 is not real-time patient data 17 may comprise graphics 21 and/or a text informing the remote clinician that the pre-disruption patient data 19 is not real-time patient data 17. In the illustrated example, the visual indication is seen to comprise both a characteristic visual appearance of the pre-disruption patient data 19, which visual appearance is distinguishably different from the characteristic visual appearance of real-time patient data 17, and graphics 21 that informs the remote clinician that the displayed patient data is not real-time patient data but pre-disruption patient data 19 corresponding to “the last known data before the disconnection”. In the example, the pre-disruption patient data 19 is presented in a grey tone on the black background of the real-time view 16, whereas the real-time patient data 17, as illustrated in FIG. 2, is presented in colour or white.

The pre-disruption data 19 that is displayed during this first period of real-time data disruption is preferably the last obtained patient data that is communicable to the remote display device 7A-D. If the real-time data disruption is caused by wireless connection problems preventing the transmission module 9A-D from transmitting patient data to the server 13 but not preventing the server 13 from communicating data to the remote display device 7A-D, the pre-disruption patient data 19 will correspond to the last patient data received by the server 13 from the transmission module 9A-D.

During the first period of real-time data disruption, the system may further be configured to display, in the real time view 16 in which the pre-disruption patient data 19 is displayed, information 22 indicating when the pre-disruption patient data 19 was obtained at the patient site 5.

The first period of real-time data disruption during which the pre-disruption patient data 19 is displayed in the real-time view 16 typically lasts for a predetermined period of time, or time window. The predetermined period of time may for example be up to 30 minutes. In some embodiments, the duration of the time window may be in the range of 2-20 minutes, preferably in the range of 2 -12 minutes and most preferably in the range of 2-8 minutes. In an exemplary embodiment, the duration of the time window is 5 minutes. In some embodiments, the duration of the time window may vary based on the type of patient data that is being displayed. For example, the duration of the time window may be determined based on the tendency of the specific type of patient data to change over time and/or a criticality of correctness of the specific type of patient data in clinical decision making. The duration of the time window may also be determined based on other parameters, such as the type of an ongoing treatment provided to the patient, the health status of the patient, etc. When the time window has elapsed, a second period of the real-time data disruption commences, during which the system is configured to prevent further display of pre-disruption patient data 19 as substitute for real-time patient data 17 in the real-time view 16 while allowing pre-disruption patient data 19 to be displayed as historical patient data 21 in a non real-time view 20 of the GUI 15.

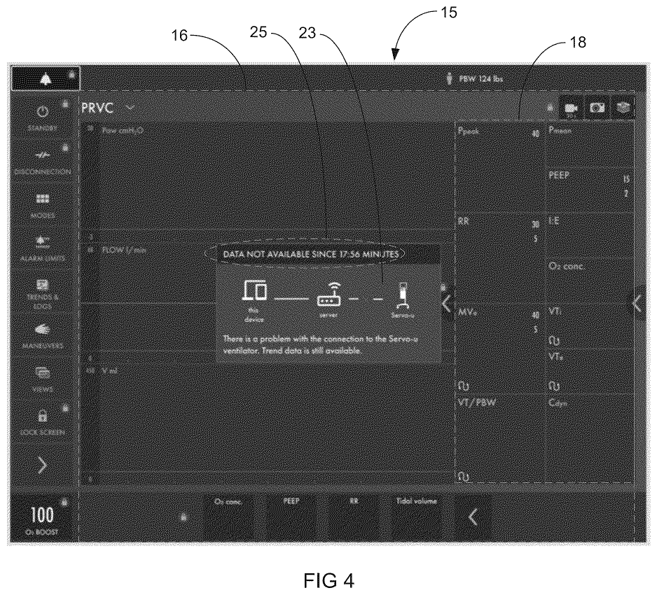

FIG. 4 illustrates the real-time view 16 of the GUI 15 during the second period of real-time data disruption. As illustrated in the drawing, the pre-disruption patient data 19 is no longer presented in the real-time data field 18. Instead, the system is configured to present graphics 23 and/or text indicating to the remote clinician that there is a real-time data disruption and that no patient data is available via the real-time view 16 of the GUI 15. As also illustrated in the drawing, the system may further be configured to display, in the real time view 16, information 25 indicating a duration of the ongoing real-time data disruption.

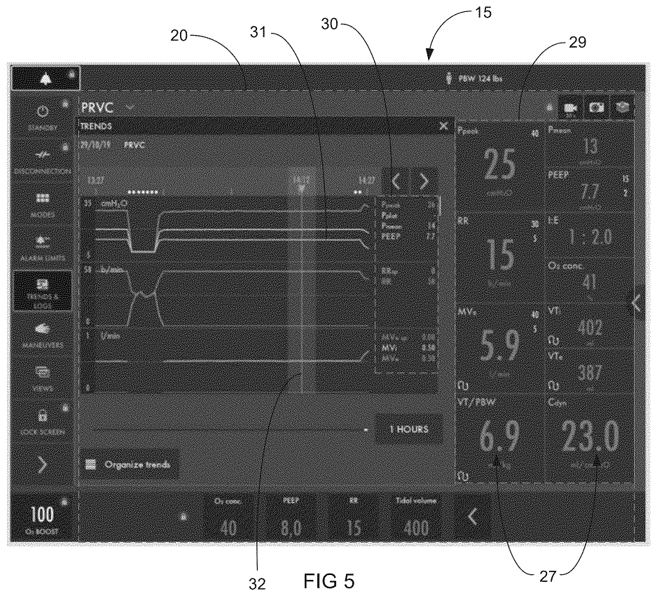

While preventing pre-disruption patient data 19 from being displayed as substitute for real-time data 17 in the real-time view 16 of the GUI 15 during this second period of real-time data disruption, the system is configured to still make pre-disruption patient data 19 available to the remote clinician via a non real-time view of the GUI. Such a non real-time view is illustrated in FIG. 5.

FIG. 5 illustrates a non real-time view 20 presenting logs 30 and trend curves 31 for historical patient data. In the non real-time view 20, pre-disruption patient data is presented as historical patient data. The historical patient data may be presented as trend curves 31 and/or numerical values. In the illustrated example, the log 30 comprises numerical values of historical patient data obtained at a point in time indicated by an indicator 32 indicating a point in time along the time axes of the trend curves 31. Furthermore, the non real-time view comprises a data field 29 for the most recently obtained historical patient data 27, corresponding to the pre-disruption patient data 19 that was made available via the real-time view 16 during the first period of real-time data disruption. To clearly indicate to the remote clinician that the most recently obtained historical patient data 27 is pre-disruption patient data which should not be confused with real-time patient data 17, the most recently obtained historical patient data 27 may be displayed with a visual appearance corresponding to the characteristic visual appearance of pre-disruption patient data 19 in the real-time view 16 (see FIG. 3), which visual appearance is distinguishably different from the characteristic visual appearance of real-time patient data 17. Of course, the non real-time view 20 may be accessible not only during real-time data disruptions but also during normal operation of the remote monitoring system when there is no disruption or loss of connectivity.

Consequently, with reference to FIGS. 2-5, the remote monitoring system is configured to display real-time patient data 17 and pre-disruption patient data 19 in a real-time view 16 of the GUI 15 of the remote display device 7A-D, and, when a real-time data disruption has lasted for more than a predetermined period of time, prevent further display of pre-disruption patient data 19 as substitute for real-time patient data 17 in the real-time view 16 while allowing pre-disruption patient data 19 to be displayed as historical patient data 21 in a non real-time view 20 of the GUI 15. In some embodiments, the system may be configured to automatically switch view of the GUI of the remote display device 7A-D from the real-time view 16 to the non real-time view 20 when the time window during which pre-disruption patient data 19 is displayed as substitute for real-time patient data 17 in the real-time view 16 has elapsed.

With reference again made to FIG. 1, in order to present patient data trends and logs for historical patient data on the remote display device 7A-D, the system server 13 may be configured to save the patient data received by the server 13 from the transmission module 9A-D on the patient site 5. The received patient data may be stored in a data storage (not shown) in the server 13, or a data storage, such as a patient data database, residing in a network node that is communicatively connected to the server 13. This enables the system to communicate historical patient data to the remote display device 7A-D via the web application in the server 13 also during real-time data disruptions caused by communication problems between the medical device 3A-D and the server 13. Another advantage is that the server 13 and/or other network nodes to which the server 13 is connected typically have higher data storage capacity than the medical device 3A-D on the patient site 5, thereby enabling more long-term trends of historical patient data to be displayed on the remote display device 7A-D.

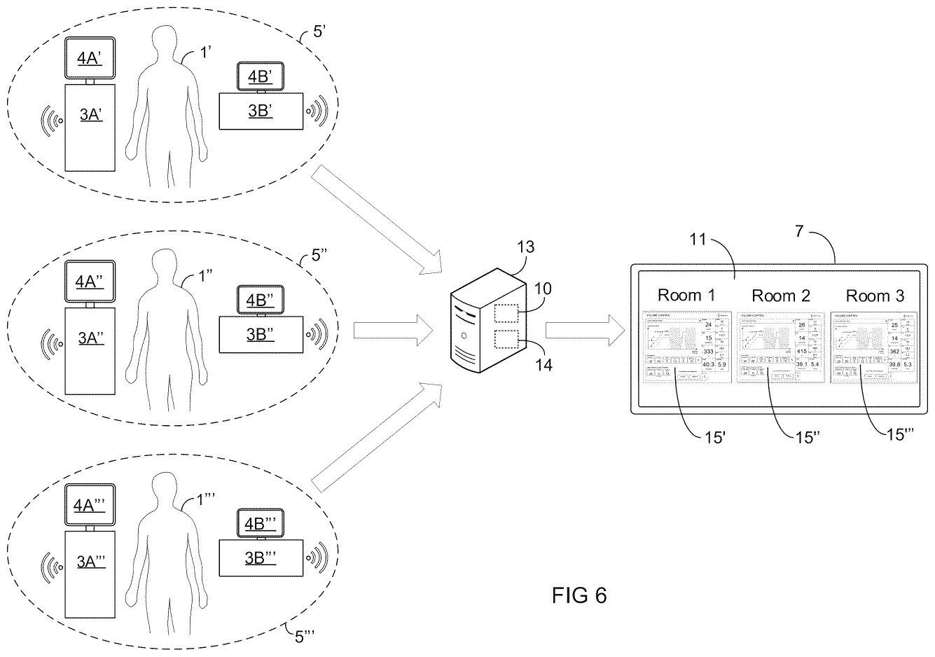

FIG. 6 illustrates an exemplary scenario where the remote monitoring system of the present disclosure is set up as a multi-patient monitoring for simultaneous remote monitoring of multiple patients 1′-1′″. In this scenario the system server 13 is configured to receive patient data obtained at a plurality of patient sites 5′-5″, each hosting a respective patient 1′-1′″ undergoing critical care treatment provided by a medical device 3A′-A′″, 3B′-B′″ located at the respective patient site. The system is configured to display patient data from the patient sites 5′-5″′ on a display 11 of a single remote display device 7, and to display real-time patient data, pre-disruption patient data and historical patient data from the patient sites 5′-5″′ in accordance with the above-described principles.

Also in accordance with the above-described principles, the multi-patient monitoring system may be configured to display replicas 15′-15′″ of the GUIs of the medical devices 3A′-A′″, 3B′-B′″ on the remote display device 7. The multi-patient monitoring system may be configured to display one GUI replica at a time, or to simultaneously display a plurality of GUI replicas replicating the GUIs of a plurality of medical devices providing critical care treatment to patients at different patient sites. In some examples, the multi-patient monitoring system may be configured to simultaneously display a plurality of GUI replicas replicating the GUIs of a plurality of medical devices of the same type, such as a plurality of breathing apparatuses 3A or a plurality of ECMO devices 3B, providing treatment to different patients. In other examples, the multi-patient monitoring system may be configured to simultaneously display a plurality of GUI replicas replicating the GUIs of a plurality of medical devices of different types, such as a breathing apparatus 3A and an ECMO device 3B, providing simultaneous treatment to the same patient.

In the illustrated example, a plurality of GUI replicas 15′-15″′ replicating the GUIs of a plurality mechanical ventilators 3A′-3A′″ providing critical care treatment to patients 1′-1′″ at a respective patient site 5′-5′″ are displayed simultaneously on the single remote display device 7. The GUI replicas 15′-15′″ are displayed side by side in a dashboard view presented on the display 11 of the remote display device 7. The GUI replicas 15′-15″′ of the dashboard view may present real-time views corresponding to the real-time view 16 illustrated in FIGS. 2-4, or non real-time views corresponding to the non real-time view 20 illustrated in FIG. 5, and to present real-time patient data 19, pre-disruption patient data 19 and historical patient data in accordance with the above described principles. This way, the remote clinician can compare any of real-time patient data 17 and historical patient data obtained at different treatments sites.

Although the remote monitoring system has been described above with reference to embodiments incorporating a server 13 for receiving, processing and forwarding patient data obtained at the patient site 5 to the remote display device 7A-D, it should be appreciated that the principles of the present disclosure could be utilized also in a remote monitoring system without such a server. For example, as illustrated by the dashed arrow 33 in FIG. 1, the patient data obtained at the patient site 5 may be transmitted directly to the remote display device 7A-D by the transmission module 9A-D at the patient site 5.

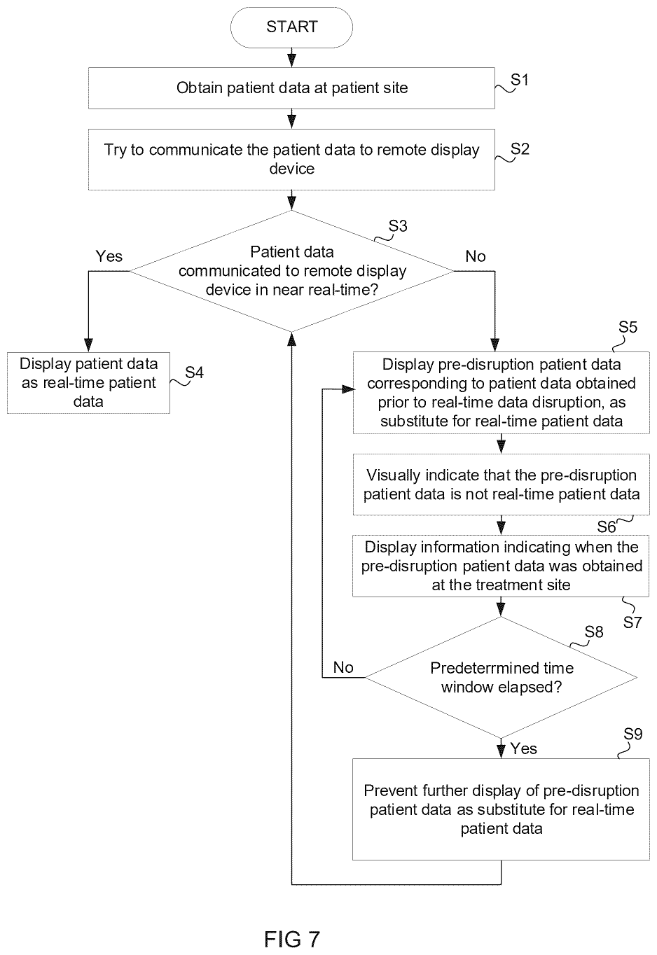

FIG. 7 is a flowchart illustrating an exemplary embodiment of a method performed in and by a system for remote monitoring of a patient located at a patient site of a healthcare facility, such as the remote monitoring system illustrated in FIG. 1 or 6. The method will hereinafter be described with simultaneous reference to the remote monitoring system of FIG. 1.

In a first step, S1, patient data is obtained by the medical device 3A-D at the patient site 5.

In a second step, S2, the system tries to communicate the obtained patient data to a remote display device 7A-D. As discussed above with reference to FIG. 1, this may, for example, be achieved by means of a transmission module 9A-D of the medical device 3A-D and a server 13 running a web application accessible by the remote display device 7A-D.

In a third step, S3, the system determines if the patient data can be communicated to the remote display device 7A-D in near real time, i.e. if patient data can be made available to a remote clinician via the remote display device 7A-D in near real time. The logic for determining if patient data can be communicated to the remote display device 7A-D typically resides in the server 13. In some embodiments, the server 13 is configured to receive patient data from the transmission module 9A-D according to a predetermined communication protocol, whereby the server 13 may conclude that patient data cannot be communicated in near real-time to the remote display device if patient data is not received from the transmission module in accordance with the predetermined communication protocol.

If it is determined that patient data can be communicated to the remote display device 7A-D in near real-time in step S3, the method proceeds to step S4 in which the patient data is displayed and presented on the remote display device 7A-D as real-time patient data 17.

If, on the other hand, it is determined that patient data cannot be communicated to the remote display device 7A-D in near real time in step S3, a real-time data disruption has occurred and the method proceeds to step S5.

In step S5, pre-disruption patient data 19 corresponding to patient data obtained prior to the real-time data disruption is displayed on the remote display device 7A-D as substitute for real-time patient data 17. As, discussed above with reference to FIG. 3, this may be achieved by replacing real-time patient data 17 with pre-disruption patient data 19 in one or more real-time data fields 18 of the GUI 15 of the remote display device 7A-D.

In step S6, which is typically performed in parallel with step S5, a visual indication indicating to a user of the remote display device 7A-D that the pre-disruption patient data 19 is not real-time data 17 is displayed together with the pre-disruption patient data on the remote display device 7A-D. As discussed above with reference to FIG. 3, the visual indication may be a characteristic visual appearance of the pre-disruption patient data and/or graphics or text displayed in association with the pre-disruption patient data.

In step S7, which is also typically performed in parallel with step S5, information indicating when the displayed pre-disruption patient data was obtained at the patient site 5 is displayed on the remote display device 7A-D. In an exemplary embodiment, the server 13 may be configured to keep track of when the last package of patient data was received from the transmission module 9A-D, and cause display of the time elapsed since reception of the last patient data package as an indicator of when the displayed pre-disruption patient data was obtained at the patient site.

In step S8, the system determines if a predetermined period of time, or time window, has elapsed since the real-time data disruption occurred. This determination may also, in some embodiments, be performed by the server 13 based on the time elapsed since reception of the last patient data package from the transmission module.

If the time window has not elapsed, the method returns to step S5 and so the pre-disruption patient data 19 will be displayed as substitute for real-time patient data 17 for the duration of the time window.

If the time window has elapsed, the method proceeds to step S9 in which further display of pre-disruption patient data as substitute for real-time patient data is prevented. As discussed above with reference to FIG. 5, the system may still, however, be configured to provide access to pre-disruption patient data presented as most recently obtained historical patient data 27 in a non real-time view 20 of the GUI 15 presented on the remote display device 7A-D.

As should be appreciated by any person skilled in the art in view of the foregoing description, the proposed principles for remote near real-time monitoring of a patient may be implemented in many different ways. The principles are not limited to any particular type of network architecture or topology and modifications and variations are possible within the scope of the appended claims.

1. A system for remote monitoring of a patient located at a patient site of a healthcare facility, the system being configured to communicate patient data obtained by a medical device collocated with the patient at the patient site to a remote display device, and to display the patient data as real-time patient data on a display of the remote display device, wherein the system is configured to:
when the patient data can be communicated to the remote display device in near real time:
display the patient data as real-time patient data on the display of the remote display device, and
when a real-time data disruption during which the patient data cannot be communicated to the remote display device in near real-time occurs:
display pre-disruption patient data corresponding to patient data obtained prior to the real-time data disruption on the display of the remote display device, as substitute for real-time patient data, and
visually indicate on the display of the remote display device that the pre-disruption patient data is not real-time patient data.

2. The system of claim 1, wherein the system is configured to display, together with the pre-disruption patient data, information indicating when the pre-disruption patient data was obtained at the patient site on the display of the remote display device.

3. The system of claim 1, wherein the system is configured to display the pre-disruption patient data on the display of the remote display device only during a predetermined time window.

4. The system of claim 3, wherein the system is configured to display the real-time patient data and the pre-disruption patient data in a real-time view of a graphical user interface, GUI, of the remote display device, and, when the predetermined time window has elapsed, prevent further display of pre-disruption patient data as substitute for real-time patient data in the real-time view while allowing pre-disruption patient data to be displayed as historical patient data in a non real-time view of the GUI.

5. The system of claim 4, wherein the GUI of the remote display device is a replica of a GUI associated with the medical device.

6. The system of claim 4, wherein the system comprises a server configured to receive the patient data obtained at the patient site and communicate the patient data to the remote display device for display as real-time patient data.

7. The system of claim 6, wherein the system is configured to save the received patient data in the server and, upon a real-time data disruption preventing the server from receiving patient data from the patient site but allowing the server to communicate with the remote display device, to communicate the saved patient data to the remote display device for display as historical patient data in the non real-time view of the GUI.

8. The system of claim 6, wherein the system is configured to
receive, in the server, patient data obtained at a plurality of patient sites, each hosting a respective patient undergoing critical care treatment provided by a medical device, and
simultaneously display patient data obtained at the plurality of patient sites as real-time patient data on a display of a single remote display device communicatively connected to the server.

9. The system of claim 1, wherein the medical device is a mechanical ventilator for providing respiratory treatment to the patient, an extracorporeal membrane oxygenation, ECMO, device for providing ECMO treatment to the patient, a bedside patient monitoring system, or a wearable medical device configured to be worn on the body of the patient.

10. A method in a system for remote monitoring of a patient located at a patient site of a healthcare facility, wherein the system is configured to communicate patient data obtained by a medical device collocated with the patient at the patient site to a remote display device, and to display the patient data as real-time patient data on a display of the remote display device, the method comprising:
when the patient data can be communicated to the remote display device in near real time:
displaying the patient data as real-time patient data on the display of the remote display device, and
when a real-time data disruption during which the patient data cannot be communicated to the remote display device in near real-time occurs:
displaying, on the display of the remote display device, pre-disruption patient data corresponding to patient data obtained prior to the real-time data disruption, as substitute for real-time patient data, and
visually indicating on the display of the remote display device that the pre-disruption patient data is not real-time patient data.

11. The method of claim 10, further comprising the step of displaying on the display of the remote display device, together with the pre-disruption patient data, information indicating when the pre-disruption patient data was obtained at the patient site.

12. The method of claim 10, wherein the pre-disruption patient data is displayed on the display of the remote display device only during a predetermined time window.

13. The method of claim 12, wherein the real-time patient data and the pre-disruption patient data is displayed in a real-time view of a GUI of the remote display device, the method comprising the steps of, when the predetermined time window has elapsed, preventing further display of pre-disruption patient data as substitute for real-time patient data in the real-time view while allowing pre-disruption patient data to be displayed as historical patient data in a non real-time view of the GUI.

14. The method of claim 13, wherein the GUI of the remote display device is a replica of a GUI associated with the medical device.

15. The method of claim 10, further comprising the steps of
receiving, in a server, the patient data obtained at the patient site, and
communicating the patient data to the remote display device for display as real-time patient data.

16. The method of claim 15, comprising the steps of:
saving the received patient data in the server and,
upon a real-time data disruption preventing the server from receiving patient data from the patient site but allowing the server to with the remote display device, communicating the saved patient data to the remote display device for display as historical patient data in the non real-time view of the GUI.

17. The method of claim 15, comprising the steps of:
receiving, in the server, patient data obtained at a plurality of patient sites, each hosting a respective patient undergoing critical care treatment provided by a medical device, and
simultaneously displaying patient data obtained at the plurality of patient sites as real-time patient data on a display of a single remote display device communicatively connected to the server.

18. (canceled)
